SEQUENCE LISTING (2 sequences)

SEQ ID NO: 1 - protein, 15 aa, synthetic construct
  SITE 3: note = X is 1-naphthylalanine (1Nal)
  SITE 7: note = X is homoarginine (HArg)
  SITE 13: note = X is hydroxyproline (HyP)
CPXDCMXDWSTPXWC

SEQ ID NO: 2 - protein, 15 aa, synthetic construct
  SITE 3: note = X is 1-naphthylalanine (1Nal)
  SITE 13: note = X is hydroxyproline (HyP)
CPXDCMKDWSTPXWC

(19) United States
(12) Patent Application Publication
(10) Pub. No.: US 2024/0398965 A1
(43) Pub. Date: Dec. 5, 2024

(54) BICYCLIC PEPTIDE LIGAND DRUG CONJUGATES

(71) Applicant: BicycleTx Limited, Cambridge (GB)

(72) Inventors: Paul John Beswick, Cambridge (GB); Gemma Elizabeth Mudd, Cambridge (GB)

(73) Assignee: BicycleTx Limited, Cambridge (GB)

(21) Appl. No.: 18/698,562

(22) PCT Filed: Oct. 6, 2022
(86) PCT No.: PCT/GB2022/052524

§ 371 Date: Apr. 4, 2024

(30) Foreign Application Priority Data
Oct. 6, 2021 (GB) 2114279.9

(51) Int. Cl. A61K 47/64; A61P 35/00

(52) U.S. Cl. CPC A61K 47/64; A61P 35/00

(57) ABSTRACT

The present invention relates to drug conjugates comprising two cytotoxic agents conjugated to a peptide ligand. The invention also relates to pharmaceutical compositions comprising said drug conjugates and to the use of said drug conjugates in preventing, suppressing or treating diseases, such as those which may be alleviated by cell death, in particular diseases characterised by defective cell types, proliferative disorders such as cancer and autoimmune disorders such as rheumatoid arthritis.

### FIELD OF THE INVENTION

The present invention relates to drug conjugates comprising two cytotoxic agents conjugated to a peptide ligand. The invention also relates to pharmaceutical compositions comprising said drug conjugates and to the use of said drug conjugates in preventing, suppressing or treating diseases, such as those which may be alleviated by cell death, in particular diseases characterised by defective cell types, proliferative disorders such as cancer and autoimmune disorders such as rheumatoid arthritis.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene).

### SUMMARY OF THE INVENTION

According to a first aspect of the invention, there is provided a drug conjugate comprising:
(i) a peptide ligand comprising at least three reactive groups, separated by at least two loop sequences, and a non-aromatic molecular scaffold which forms covalent bonds with the reactive groups of the peptide ligand such that at least two polypeptide loops are formed on the molecular scaffold; and
(ii) two cytotoxic agents conjugated to said peptide ligand.

According to a second aspect of the invention, there is provided a pharmaceutical composition comprising a drug conjugate as defined herein in combination with one or more pharmaceutically acceptable excipients.

According to a further aspect of the invention, there is provided a drug conjugate as defined herein for use in preventing, suppressing or treating diseases, such as those which may be alleviated by cell death, in particular diseases characterised by defective cell types, proliferative disorders such as cancer and autoimmune disorders such as rheumatoid arthritis.

### BRIEF DESCRIPTION OF THE FIGURES

FIG. 1: Tumor volume traces after administering BCY8243 to female Balb/c nude mice bearing NCI-H292 xenograft. Error bars represent standard error of the mean (SEM).

FIG. 2: Tumor volume traces after administering BCY8391 to female Balb/c nude mice bearing NCI-H292 xenograft. Error bars represent standard error of the mean (SEM).

FIG. 3: Tumor volume traces after administering BCY8242 to female Balb/c nude mice bearing NCI-H292 xenograft. Error bars represent standard error of the mean (SEM).

### DETAILED DESCRIPTION OF THE INVENTION

According to a first aspect of the invention, there is provided a drug conjugate comprising:
(i) a peptide ligand comprising at least three reactive groups, separated by at least two loop sequences, and a non-aromatic molecular scaffold which forms covalent bonds with the reactive groups of the peptide ligand such that at least two polypeptide loops are formed on the molecular scaffold; and
(ii) two cytotoxic agents conjugated to said peptide ligand.

In one embodiment, said peptide ligand is specific for Nectin-4.

Nectin-4 is a surface molecule that belongs to the nectin family of proteins, which comprises 4 members. Nectins are cell adhesion molecules that play a key role in various biological processes such as polarity, proliferation, differentiation and migration, for epithelial, endothelial, immune and neuronal cells, during development and adult life. They are involved in several pathological processes in humans. They are the main receptors for poliovirus, herpes simplex virus and measles virus. Mutations in the genes encoding Nectin-1 (PVRL1) or Nectin-4 (PVRL4) cause ectodermal dysplasia syndromes associated with other abnormalities. Nectin-4 is expressed during foetal development. In adult tissues its expression is more restricted than that of other members of the family. Nectin-4 is a tumour-associated antigen in 50%, 49% and 86% of breast, ovarian and lung carcinomas, respectively, mostly on tumours of bad prognosis. Its expression is not detected in the corresponding normal tissues. In breast tumours, Nectin-4 is expressed mainly in triple-negative and ERBB2+ carcinomas. In the serum of patients with these cancers, the detection of soluble forms of Nectin-4 is associated with a poor prognosis. Levels of serum Nectin-4 increase during metastatic progression and decrease after treatment. These results suggest that Nectin-4 could be a reliable target for the treatment of cancer. Accordingly, several anti-Nectin-4 antibodies have been described in the prior art. In particular, Enfortumab Vedotin (ASG-22ME) is an antibody-drug conjugate (ADC) targeting Nectin-4 and is currently clinically investigated for the treatment of patients suffering from solid tumours.

In one embodiment, the Nectin-4 is mammalian Nectin-4. In a further embodiment, the mammalian Nectin-4 is human Nectin-4.

In one embodiment, said peptide ligand is specific for Nectin-4 and said loop sequences comprise 3 or 9 amino acid acids.

In a further embodiment, said peptide ligand is specific for Nectin-4 and said loop sequences comprise three cysteine residues separated by two loop sequences one of which consists of 3 amino acids and the other of which consists of 9 amino acids. Thus, in one embodiment, said peptide ligand is specific for Nectin-4 and has a core sequence of:

(SEQ ID NO: 1)
CP(1Nal)(dD)CM(HArg)DWSTP(HyP)WC,

wherein 1Nal represents 1-naphthylalanine, HArg represents homoarginine and HyP represents hydroxyproline.

In an alternative embodiment, said peptide ligand is specific for Nectin-4 and has a core sequence of:

(SEQ ID NO: 2)
CP(1Nal)(dD)CMKDWSTP(HyP)WC,

wherein 1Nal represents 1-naphthylalanine and HyP represents hydroxyproline.

Unless defined otherwise, all technical and scientific terms used herein have the same meaning as commonly understood by those of ordinary skill in the art, such as in the arts of peptide chemistry, cell culture and phage display, nucleic acid chemistry and biochemistry.

Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within the bicyclic peptides of the invention, cysteine residues (Cᵢ, Cᵢᵢ and Cᵢᵢᵢ) are omitted from the numbering as they are invariant, therefore, the numbering of amino acid residues within a selected bicyclic peptide of the invention is referred to as below:
—Cᵢ-P₁-1Nal₂-dD₃-Cᵢᵢ-M₄-HArg₅-D₆-W₇-S₈-T₉-P₁₀-HyP₁₁-W₂-Cᵢᵢᵢ- (SEQ ID NO: 1).

For the purpose of this description, all bicyclic peptides are assumed to be cyclised with 1,1,1″-(1,3,5-triazinane-1,3,5-triyl)triprop-2-en-1-one (TATA) and yielding a tri-substituted structure. Cyclisation with TATA occurs on Cᵢ, Cᵢᵢ, and Cᵢᵢᵢ.

### Molecular Format

N- or C-terminal extensions to the bicycle core sequence are added to the left or right side of the sequence, separated by a hyphen. For example, an N-terminal βAla-Sar₁₀-Ala tail would be denoted as:

(SEQ ID NO: X)
BAla-Sar10-A-.

### Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

### Peptide Ligands

A peptide ligand, as referred to herein, refers to a peptide, peptidic or peptidomimetic covalently bound to a molecular scaffold. Typically, such peptides, peptidics or peptidomimetics comprise a peptide having natural or non-natural amino acids, two or more reactive groups (i.e. cysteine residues) which are capable of forming covalent bonds to the scaffold, and a sequence subtended between said reactive groups which is referred to as the loop sequence, since it forms a loop when the peptide, peptidic or peptidomimetic is bound to the scaffold. In the present case, the peptides, peptidics or peptidomimetics comprise at least three cysteine residues (referred to herein as Cᵢ, Cᵢᵢ and Cᵢᵢᵢ), and form at least two loops on the scaffold.

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure to develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity. Certain peptide ligands of the invention demonstrate good selectivity over other receptor subtypes. For example, when the bicyclic peptide is specific for Nectin-4, said bicyclic peptide will be ideally selective for Nectin-4 over other Nectin sub-types.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO—), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the invention contain an amine function, these may form quaternary ammonium salts, for example by reaction with an alkylating agent according to methods well known to the skilled person. Such quaternary ammonium compounds are within the scope of the compounds of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with one or more replacement amino acids, such as an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal residue is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal residue is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, CD-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In one embodiment, the modified derivative comprises removal of any amino acid residues and substitution with alanines, such as D-alanines. This embodiment provides the advantage of identifying key binding residues and removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as 177Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the EphA2 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Reactive Groups

The molecular scaffold of the invention may be bonded to the polypeptide via functional or reactive groups on the polypeptide. These are typically formed from the side chains of particular amino acids found in the polypeptide polymer.

The reactive groups are groups capable of forming a covalent bond with the molecular scaffold. Typically, the reactive groups are present on amino acid side chains on the peptide. Examples are lysine, arginine, histidine and sulfur containing groups such as cysteine, methionine as well as analogues such as selenocysteine.

In one embodiment, said reactive groups comprise cysteine and/or (S)-2-amino-3-(methylamino)propanoic acid (Dap(Me)). In a further embodiment, all three reactive groups comprise cysteine. In an alternative embodiment, two reactive groups comprise (S)-2-amino-3-(methylamino)propanoic acid (Dap(Me)) and one reactive group comprises cysteine.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers. In another embodiment of the invention, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved. In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis, et al., Nat Chem Biol 2009, 5 (7), 502-7.

It will be appreciated that the looped bicyclic peptide structure is further attached to the molecular scaffold via at least one thioether linkage. The thioether linkage provides an anchor during formation of the bicyclic peptides. In one embodiment, there is only one such thioether linkage. In further embodiments, there is one such thioether linkage and two amino linkages. In further embodiments, there is one such thioether linkage and two alkylamino linkages. Suitably, the thioether linkage is a central linkage of the bicyclic or polycyclic peptide conjugate, i.e. in the peptide sequence two residues (e.g. diaminopropionic acid residues) forming the amino linkages in the peptide are spaced from and located on either side of the amino acid residue (e.g. lysine) forming the thioether linkage. Suitably, the looped peptide structure is therefore a bicyclic peptide conjugate having a central thioether linkage and two peripheral amino linkages. In some embodiments, placement of the thioether bond can be N-terminal or C-terminal to two N-alkylamino linkages.

In one embodiment, the reactive groups comprise one cysteine residue and two (S)-2-amino-3-(methylamino)propanoic acid (Dap(Me)) or N-beta-C-₁₋₄ alkyl-L-2, 3-diaminopropionic acid (N-AlkDap) residues.

### Non-Aromatic Molecular Scaffold

References herein to the term “non-aromatic molecular scaffold” refer to any molecular scaffold as defined herein which does not contain an aromatic (i.e. unsaturated) carbocyclic or heterocyclic ring system.

Suitable examples of non-aromatic molecular scaffolds are described in Heinis et al (2014) Angewandte Chemie, International Edition 53(6) 1602-1606.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

An example of an αβ unsaturated carbonyl containing compound is 1,1′,1″-(1,3,5-triazinane-1,3,5-triyl)triprop-2-en-1-one (TATA) (Angewandte Chemie, International Edition (2014), 53(6), 1602-1606).

### Cytotoxic Agents

Suitable examples of cytotoxic agents include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin, calicheamycins, and others.

In one embodiment of the invention, the cytotoxic agent is selected from maytansinoids (such as DM1) or monomethyl auristatins (such as MMAE).

DM1 is a cytotoxic agent which is a thiol-containing derivative of maytansine and has the following structure:

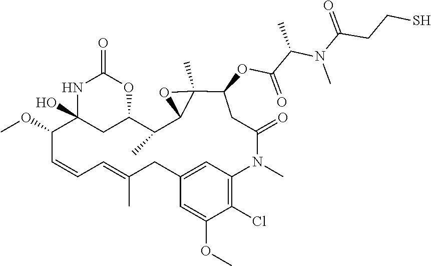 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@@H](OC)/C=C\C=C(/C)C2]

(S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide) (monomethyl auristatin E; MMAE) is a synthetic antineoplastic agent and has the following structure:

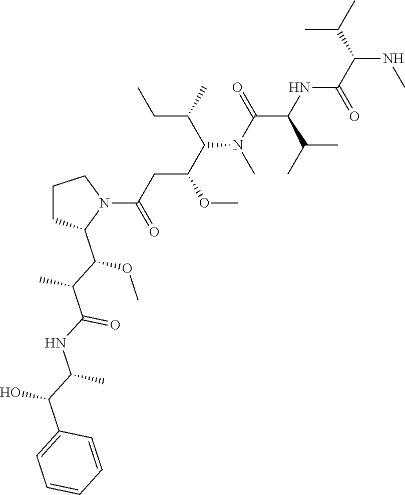

In one yet further particular embodiment of the invention, the cytotoxic agent is selected from MMAE.

In one embodiment, the cytotoxic agent is linked to the bicyclic peptide by a cleavable bond, such as a disulphide bond or a protease sensitive bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

In one embodiment, the cytotoxic agent and linker is selected from any combinations of those described in WO 2016/067035 (the cytotoxic agents and linkers thereof are herein incorporated by reference).

In one embodiment, the linker between said cytotoxic agent and said bicyclic peptide comprises one or more amino acid residues. Examples of suitable amino acid residues as suitable linkers include Ala, Cit, Lys, Trp and Val. In a further embodiment, the linker between said cytotoxic agent and said bicyclic peptide comprises a Val-Cit moiety. In a further embodiment, the linker between said cytotoxic agent and said bicyclic peptide comprises a β-Ala moiety.

In one embodiment, the linker between said cytotoxic agent and said bicyclic peptide comprises p-aminobenzylcarbamate (PABC).

In one embodiment, the linker between said cytotoxic agent and said bicyclic peptide comprises a glutaryl moiety.

In one embodiment, the linker between said cytotoxic agent and said bicyclic peptide comprises N-(bis aminopropyl) glycine (BAPG).

In one embodiment, the linker between said cytotoxic agent and said bicyclic peptide comprises one or more (e.g. 10) sarcosine (Sar) residues.

In a further embodiment, the linker between said cytotoxic agent and said bicyclic peptide comprises:
(i) a -Sar₁₀-βAla-BAPG-(glutaryl-Val-Cit-PABC)₂ linker (i.e. the resultant bicyclic peptide drug conjugate comprises a Bicyclic peptide-Sar₁₀-βAla-BAPG-(Glu-Val-Cit-PABC-MMAE)₂ moiety); or
(ii) a -BAPG-(Sar₁₀-βAla-glutaryl-Val-Cit-PABC)₂ linker (i.e. the resultant bicyclic peptide drug conjugate comprises a Bicyclic peptide-BAPG-(Sar₁₀-βAla-Glu-Val-Cit-PABC-MMAE)₂ moiety); or
(iii) a (Sar₁₀-βAla-glutaryl-Val-Cit-PABC)₂ linker (i.e. the resultant bicyclic peptide drug conjugate comprises a Bicyclic peptide-(Sar₁₀-βAla-Glu-Val-Cit-PABC-MMAE)₂ moiety, wherein the Sar₁₀ moiety is attached to said bicycle at two independent attachment points).

In one embodiment, the bicyclic peptide ligand is specific for Nectin-4, the cytotoxic agent is MMAE, the number of MMAE moieties is two and the drug conjugate comprises a compound of BCY8391:

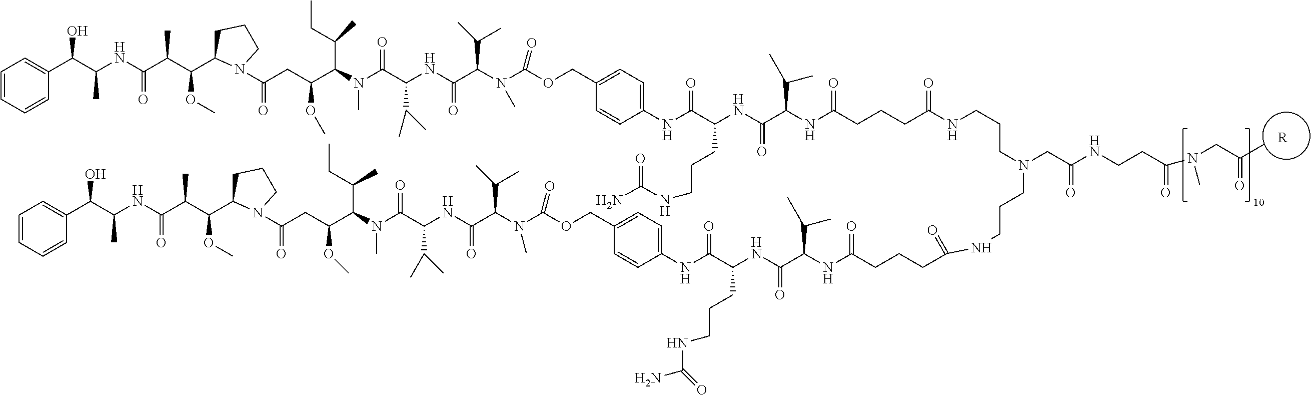

wherein R represents SEQ ID NO: 1 such that R is linked via the N-terminus of said peptide.

Data is presented herein in FIG. 2 and Tables 1 and 2 which shows that BCY8391 demonstrated significant anti-tumor activity and in particular showed comparable tumor inhibitor 0 effect and regressed the tumors potently.

In an alternative embodiment, the bicyclic peptide ligand is specific for Nectin-4, the cytotoxic agent is MMAE, the number of MMAE moieties is two and the drug conjugate comprises a compound of BCY8242:

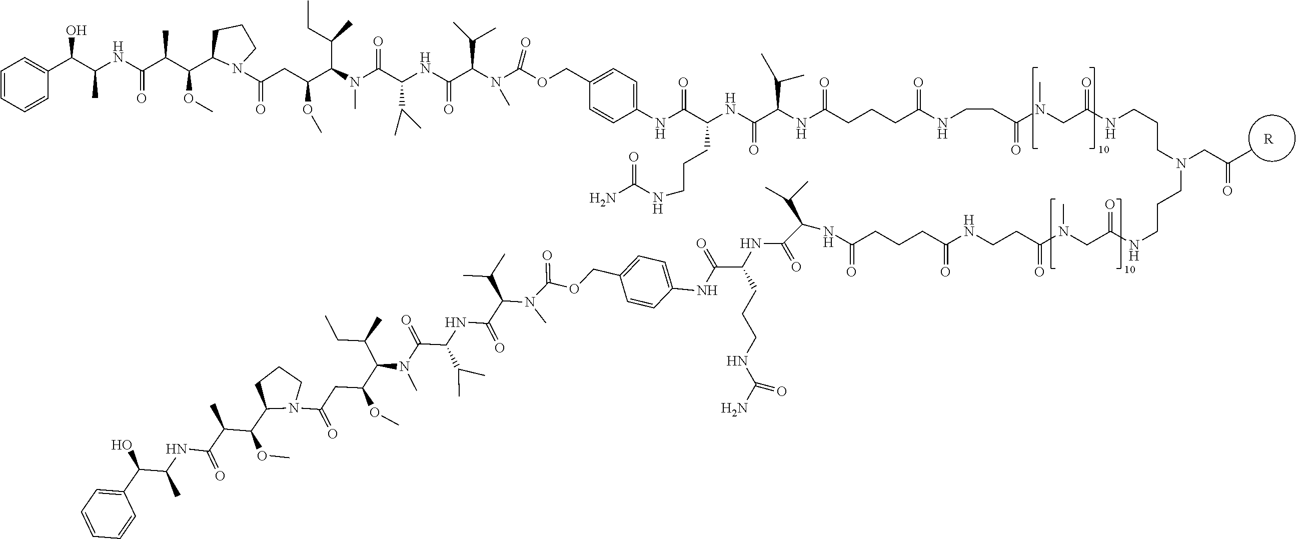

wherein R represents SEQ ID NO: 1 such that R is linked via the N-terminus of said peptide.

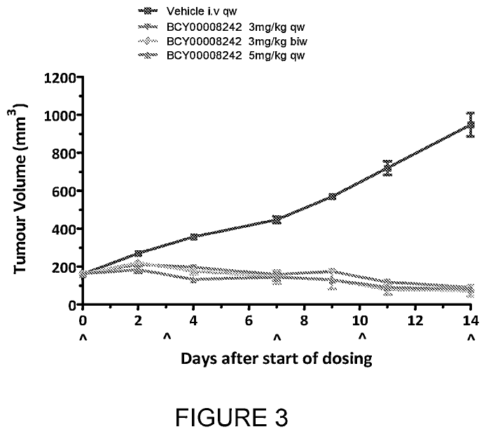

Data is presented herein in FIG. 3 and Tables 3 and 4 which shows that BCY8242 produced significant antitumor activity.

In an alternative embodiment, the bicyclic peptide ligand is specific for Nectin-4, the cytotoxic agent is MMAE, the number of MMAE moieties is two and the drug conjugate comprises a compound of BCY8243:

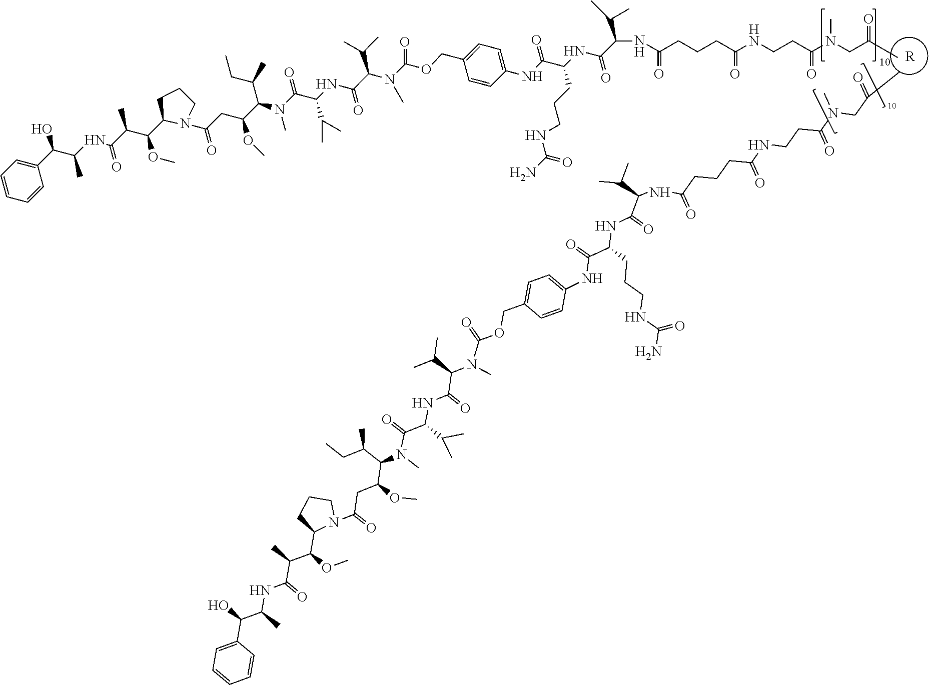

wherein R represents SEQ ID NO: 2 such that R is linked via the N-terminus and the Lys residue of said peptide.

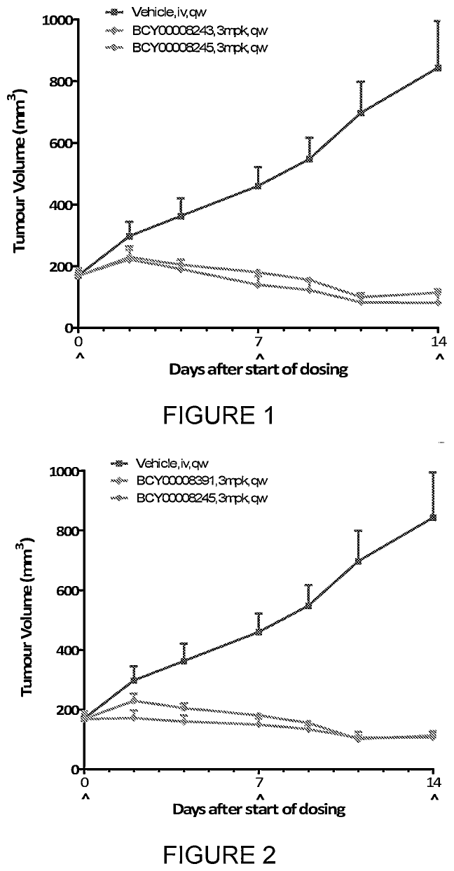

Data is presented herein in FIG. 1 and Tables 1 and 2 which shows that BCY8243 demonstrated significant anti-tumor activity and in particular showed comparable tumor inhibitory effect and regressed the tumors potently.

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand or a drug conjugate as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cylcosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

By virtue of the presence of the cytotoxic agent, the drug conjugates of the invention have specific utility in the treatment of diseases which may be alleviated by cell death. Examples of suitable diseases include diseases characterised by defective cell types, proliferative disorders such as cancer and autoimmune disorders such as rheumatoid arthritis.

By virtue of the presence of the cytotoxic agent coupled to a cancer cell binding bicyclic peptide, the bicyclic peptides of the invention have specific utility in the treatment of cancer. Thus, according to a further aspect of the invention, there is provided a drug conjugate as defined herein for use in preventing, suppressing or treating cancer (such as a tumour). According to a further aspect of the invention, there is provided a method of preventing, suppressing or treating cancer (such as a tumour), which comprises administering to a patient in need thereof a drug conjugate as defined herein.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is selected from: breast cancer, lung cancer, gastric cancer, pancreatic cancer, prostate cancer, liver cancer, glioblastoma and angiogenesis.

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

### Abbreviations

1 NAI 1-Naphthylalanine
HyP Hydroxyproline
HArg HomoArginine
β-Ala β-Alanine
Sar Sarcosine, such that Sarₓ represents x Sar residues

### Materials and Methods

### Peptide Synthesis

Peptides were synthesized by solid phase synthesis. Rink Amide MBHA Resin was used. To a mixture containing Rink Amide MBHA (0.4-0.45 mmol/g) and Fmoc-Cys(Trt)-OH (3.0 eq) was added DMF, then DIC (3 eq) and HOAt (3 eq) were added and mixed for 1 hour. 20% piperidine in DMF was used for deblocking. Each subsequent amino acid was coupled with 3 eq using activator reagents, DIC (3.0 eq) and HOAT (3.0 eq) in DMF. The reaction was monitored by ninhydrin color reaction or tetrachlor color reaction. After synthesis completion, the peptide resin was washed with DMF×3, MeOH×3, and then dried under N2 bubbling overnight. The peptide resin was then treated with 92.5% TFA/2.5% TIS/2.5% EDT/2.5% H2O for 3 h. The peptide was precipitated with cold isopropyl ether and centrifuged (3 min at 3000 rpm). The pellet was washed twice with isopropyl ether and the crude peptide was dried under vacuum for 2 hours and then lyophilised. The lyophilised powder was dissolved in of ACN/H2O (50:50), and a solution of 100 mM TATA in ACN was added, followed by ammonium bicarbonate in H₂O (1M) and the solution mixed for 1 h. Once the cyclisation was complete, the reaction was quenched with 1M aq. Cysteine hydrochloride (10 eq relative to TATA), then mixed and left to stand for an hour. The solution was lyophilised to afford crude product. The crude peptide was purified by Preparative HPLC and lyophilized to give the product

All amino acids, unless noted otherwise, were used in the L-configurations.

### Preparation of Drug Conjugates of the Invention

Reactions were monitored using LC-MS (Acquity UPLC CSH C18 column, 1.7 μm, 2.1×30 mm; acetonitrile/water/HCOOH containing buffers and 15 to 60% acetonitrile gradient elution over 10 min). Products from the reactions were purified using RP-HPLC (Gemini C18—semi prep column, 5 μm, 110 Å, 250×10 mm; acetonitrile/water/TFA containing buffers and 20 to 80% acetonitrile gradient elution over 20 min).

### Preparation of BCY8391

### (i) Preparation of Fmoc-BAPG-TFP

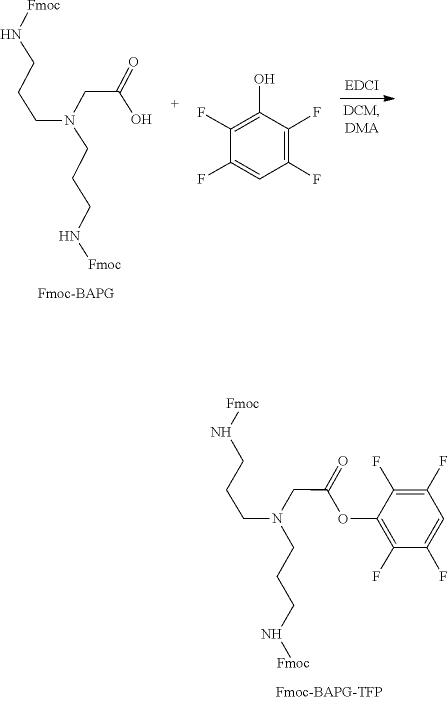

To a solution of Fmoc-BAPG (200 mg, 316 μmol) was dissolved in DMA (4.5 mL) and DCM (1.5 mL), to this solution 2,3,5,6-tetrafluorophenol (157 mg, 947 μmol) and EDCl (212 mg, 1.10 mmol) were added under nitrogen atmosphere. Then the mixture was stirred at 25° C. for 16 hr. LC-MS showed Fmoc-BAPG was consumed completely and one main peak with desired mass was detected. The reaction mixture was concentrated to remove the DCM. The left solution was purified by prep-HPLC (neutral condition) to give compound Fmoc-BAPG-TFP (140 mg, 179 μmol, 56.7% yield) as a white solid. LCMS m/z=782.5 [M+H]+, RT=1.0 min

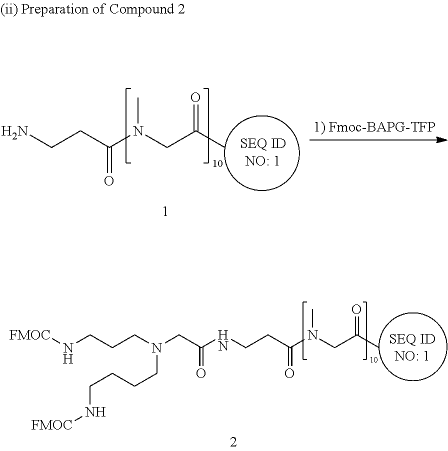

To a solution of compound 1 (50.0 mg, 16.9 μmol) in DMA (1 mL) was added DIEA (10.9 mg, 84.6 μmol, 14.7 μL) and the mixture was stirred at 25° C. for 10 min. Then Fmoc-BAPG-TFP (36.0 mg, 46.1 μmol) was added under nitrogen and the mixture was stirred at 25° C. for 2 hr. LC-MS showed compound 1 was consumed completely and one main peak with desired mass was detected. LCMS m/z=1191.0 [M+H]3+, RT=0.86 min

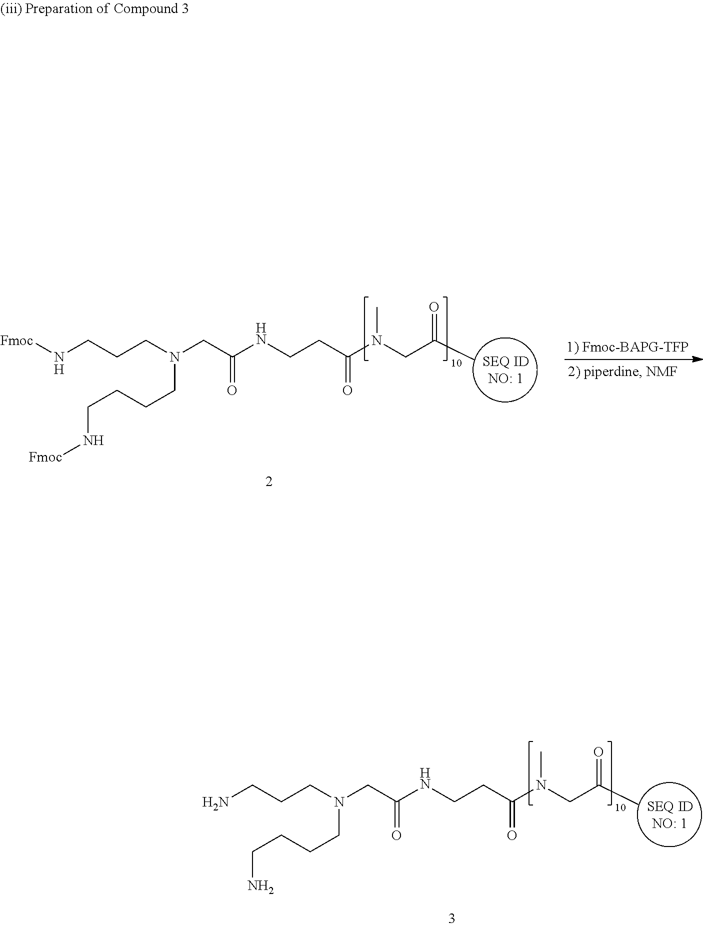

To a crude compound 2 was added Piperidine (86.2 mg, 1.01 mmol, 100 μL), then the mixture was stirred at 25° C. for 10 min. LC-MS showed compound 2 was consumed completely and one main peak with desired mass was detected. The reaction mixture was diluted with 0.5 mL of water and purified by prep-HPLC (neutral condition) to give compound 3 (40.0 mg, 12.8 μmol, 76.2% yield) as a white solid. LCMS m/z=782.4 [M+H]3+, RT=0.72 min

### (iv) Preparation of BCY8391

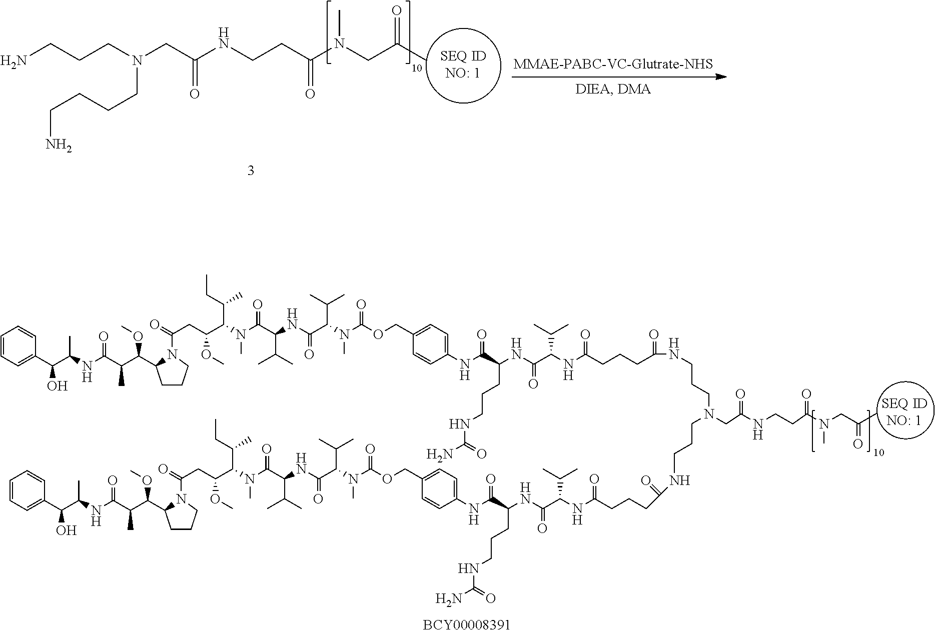

To a solution of compound 3 (30.0 mg, 9.60 μmol) in DMA (3 mL) was added DIEA (24.8 mg, 192 μmol, 33.4 μL) with stirring for 10 min under nitrogen atmosphere. Then MMAE-PABC-VC-Glutaryl-NHS (64.1 mg, 48.0 μmol) was added to the DMA solution and the mixture was further stirred at 15° C. for additional 4 hr. LC-MS showed desired mass was detected. HPLC showed the purity of desired product. The reaction mixture was diluted with 1 mL of water and purified by prep-HPLC (TFA condition) to give compound BCY8391 (17.8 mg, 3.10 μmol, 32.3% yield, 96.9% purity) as a white solid. LCMS m/z=1856.0 [M+H]3+, RT=1.13 min. HPLC RT=11.47 min

### Preparation of BCY8242

BCY8242 was prepared in accordance with the following scheme:

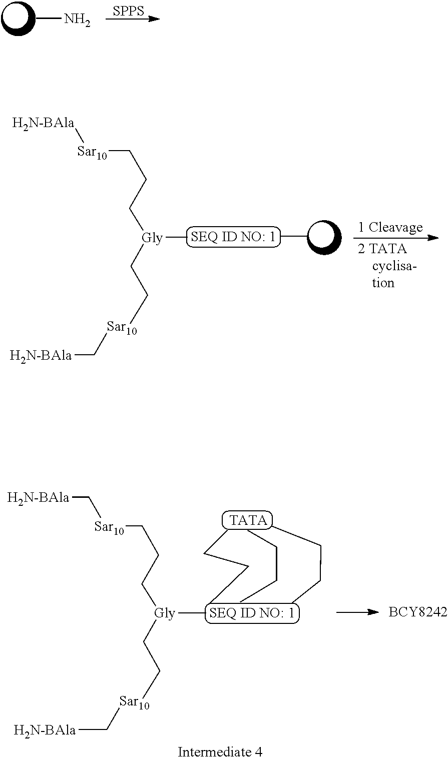

The final stage was achieved using an analogous method to that described above, starting with Intermediate 4 (80 mg) and MMAE-PABC-vc-Glutarate-NHS (50 mg). The reaction gave BCY8243 (48.7 mg, 43.9%) and 97.4% purity. LCMS m/z=1587.4, [M+H]3+, RT=1.08 min. HPLC RT=1357 min

### Preparation of BCY8243

BCY8243 was prepared in accordance with the following scheme:

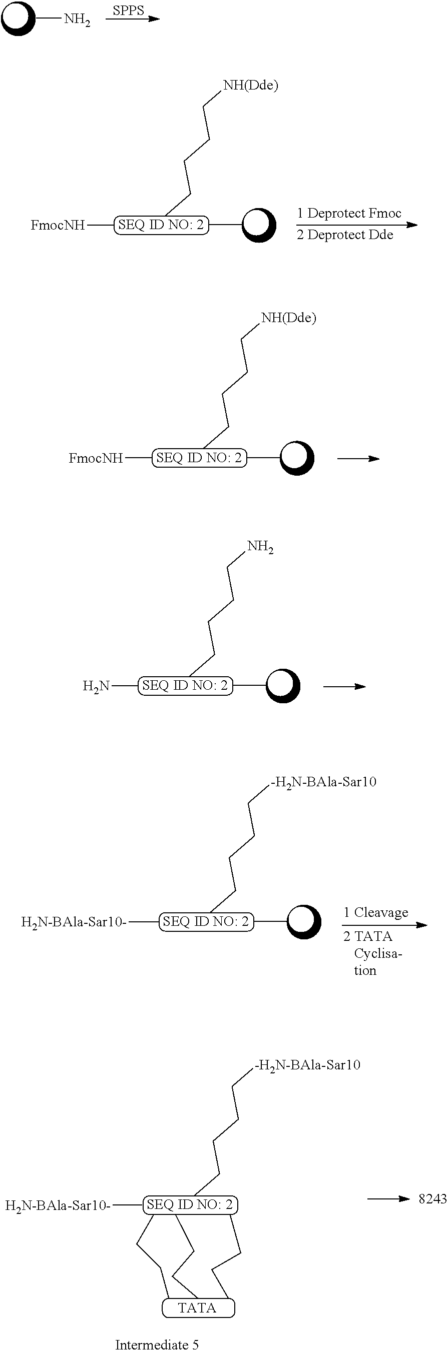

The final stage was achieved using an analogous method to that described above, starting with Intermediate 5 (70 mg) and MMAE-PABC-vc-Glutarate-NHS (50 mg). The reaction gave BCY8243 (54.6 mg, 48.46%) and 92.7% purity. LCMS m/z=1022.4, [M+H]4+, RT=1.06 min. HPLC RT=9.30 min.

### Biological Data

### Example 1: In Vivo Efficacy Study of BCY8391 and BCY8243 in the Treatment of NCI-H292 Xenograft in Balb/c Nude Mice

### 1. Study Objective

The objective of the research is to evaluate the in vivo anti-tumor efficacy of BCY8391 and BCY8243 in the treatment of NCI-H292 xenograft in Balb/c nude mice.

### 2. Experimental Design

| | | | | Dosing | | |
|---|---|---|---|---|---|---|
| | | | Dose | Volume | Dosing | |
| Group | Treatment | n | (mg/kg) | (ul/g) | Route | Schedule |
| 1 | Vehicle | 4 | — | 10 | iv | qw |
| 2 | BCY8243 | 3 | 3 | 10 | iv | qw |
| 3 | BCY8391 | 3 | 3 | 10 | iv | qw |

### 3. Materials

### 3.1 Animals and Housing Condition

### 3.1.1 Animals

Species: Mus Musculus
Strain: Balb/c nude
Age: 6-8 weeks
Sex: female
Body weight: 18-22 g
Number of animals: 43 mice plus spare
Animal supplier: Shanghai Lingchang Biotechnology Experimental Animal Co. Ltd

### 3.1.2 Housing Condition

The mice were kept in individual ventilation cages at constant temperature and humidity with 3 or 4 animals in each cage.
   Temperature: 20-26° C.
   Humidity 40-70%.
Cages: Made of polycarbonate. The size is 300 mm×180 mm×150 mm. The bedding material is corn cob, which is changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, the date received, treatment, study number, group number and the starting date of the treatment.
Animal identification: Animals were marked by ear coding.

### 3.2 Test and Positive Control Articles

Product identification: BCY8243
Manufacturer: Bicycle Therapeutics
Lot number: 1
Physical description: Lyophilised powder
Molecular weight: 6133.18
Purity: 97.70%
Package and storage condition: stored at −80° C.
Product identification: BCY8391
Manufacturer: Bicycle Therapeutics
Lot number: 1
Physical description: Lyophilised powder
Molecular weight: 5564.73
Purity: 96.90%
Package and storage condition: stored at −80° C.

### 4. Experimental Methods and Procedures

### 4.1 Cell Culture

The NCI-H292 tumor cells were maintained in vitro as a monolayer culture in RPMI-1640 medium supplemented with 10% heat inactivated fetal bovine serum at 37° C. in an atmosphere of 5% CO₂ in air. The tumor cells were routinely subcultured twice weekly by trypsin-EDTA treatment. The cells growing in an exponential growth phase were harvested and counted for tumor inoculation.

### 4.2 Tumor Inoculation

Each mouse was inoculated subcutaneously at the right flank with NCI-H292 tumor cells (10×10⁶) in 0.2 ml of PBS for tumor development. 43 animals were randomized when the average tumor volume reached 168 mm³. The test article administration and the animal numbers in each group were shown in the experimental design table. 4.3 Testing Article Formulation Preparation

| | Con. | |
|---|---|---|
| Treatment | (mg/ml) | Formulation |
| Vehicle | — | 25 mM Histidine pH 7 10% sucrose |
| BCY8243 | 1 | Dissolved 1.8 mg BCY8243 in 1759 μl vehicle |
| | | buffer |
| BCY8243 | 0.3 | Dilute 240 μl 1 mg/ml BCY8242 stock with 560 μl |
| | | vehicle buffer |
| BCY8391 | 1 | Dissolved 2.06 mg BCY8391 in 40 μl DMSO, then |
| | | dilute with 1960 μl 10% Kolliphor, 50 mM Hepes |
| | | pH 7 |
| BCY8391 | 0.3 | Dilute 240 μl 1 mg/ml BCY8391 stock with 560 μl |
| | | 10% Kolliphor, 50 mM Hepes pH 7 |

### 4.4 Observations

All the procedures related to animal handling, care and the treatment in the study were performed according to the guidelines approved by the Institutional Animal Care and Use Committee (IACUC) of WuXi AppTec, following the guidance of the Association for Assessment and Accreditation of Laboratory Animal Care (AAALAC). At the time of routine monitoring, the animals were checked for any effects of tumor growth and treatments on normal behavior such as mobility, food and water consumption (by looking only), body weight gain/loss, eye/hair matting and any other abnormal effect as stated in the protocol. Death and observed clinical signs were recorded on the basis of the numbers of animals within each subset.

### 4.5 Tumor Measurements and the Endpoints

The major endpoint was to see if the tumor growth could be delayed or mice could be cured. Tumor volume was measured three times weekly in two dimensions using a caliper, and the volume was expressed in mm³ using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day. TGI was calculated for each group using the formula: TGI (%)=[1−(Tᵢ−T₀)/(Vᵢ−V₀)]×100; Tᵢ is the average tumor volume of a treatment group on a given day, T₀ is the average tumor volume of the treatment group on the day of treatment start, Vi is the average tumor volume of the vehicle control group on the same day with Tᵢ, and V₀ is the average tumor volume of the vehicle group on the day of treatment start.

### 4.7 Statistical Analysis

Summary statistics, including mean and the standard error of the mean (SEM), were provided for the tumor volume of each group at each time point.

Statistical analysis of difference in tumor volume among the groups was conducted on the data obtained at the best therapeutic time point after the final dose.

A t-test was performed to compare tumor volume among groups, and when a significant. All data were analyzed using GraphPad Prism 5.0. P<0.05 was considered to be statistically significant.

### 5. Results

### 5.1 Tumor Growth Curve

Tumor growth curves are shown in FIGS. 6 and 7.

### 5.2 Tumor Volume Trace

Mean tumor volume over time in female Balb/c nude mice bearing NCI-H292 xenograft is shown in Table 1.

TABLE 1
Tumor volume trace over time

| | | Days after the start of treatment | | | | | | |
|---|---|---|---|---|---|---|---|---|
| Gr. | Treatment | 0 | 2 | 4 | 7 | 9 | 11 | 14 |
| 1 | Vehicle, qw | 168 ± 16 | 297 ± 48 | 362 ± 58 | 460 ± 62 | 548 ± 69 | 697 ± 102 | 843 ± 152 |
| 2 | BCY8243, | 168 ± 12 | 221 ± 43 | 190 ± 31 | 139 ± 28 | 122 ± 31 | 82 ± 29 | 81 ± 34 |
| | 3 mpk, qw | | | | | | | |
| 3 | BCY8391, | 168 ± 20 | 171 ± 25 | 158 ± 21 | 149 ± 20 | 133 ± 15 | 105 ± 20 | 107 ± 21 |
| | 3 mpk, qw | | | | | | | |

### 5.3 Tumor Growth Inhibition Analysis

Tumor growth inhibition rate for test articles in the NCI-H292 xenograft model was calculated based on tumor volume measurements at day 14 after the start of treatment.

TABLE 2
Tumor growth inhibition analysis

| | | Tumor | | | |
|---|---|---|---|---|---|
| | | Volume | T/C^{b} | TGI | |
| Gr | Treatment | (mm³)ª | (%) | (%) | P value |
| 1 | Vehicle, qw | 843 ± 152 | — | — | — |
| 2 | BCY8243, | 81 ± 34 | 9.6 | 113.0 | p < 0.01 |
| | 3 mpk, qw | | | | |
| 3 | BCY8391, | 107 ± 21 | 12.7 | 109.1 | p < 0.01 |
| | 3 mpk, qw | | | | |

^{a}Mean ± SEM.
^{b}Tumor Growth Inhibition is calculated by dividing the group average tumor volume for the treated group by the group average tumor volume for the control group (T/C).

### 6. Results Summary and Discussion

In this study, the therapeutic efficacy of BCY8391 and BCY8243 in the NCI-H292 xenograft model was evaluated. The measured tumor volume of all treatment groups at various time points are shown in FIGS. 1 and 2 and Tables 1 and 2.

The mean tumor size of vehicle treated mice reached 843 mm³ on day 14.

BCY8391 and BCY8243 at 3 mg/kg showed significant anti-tumor activity and in particular showed comparable tumor inhibitory effect and regressed the tumors potently.

In this study, all mice maintained the bodyweight well.

### Example 2: In Vivo Efficacy Study of BCY8242 in the Treatment of NCI-H292 Xenograft in Balb/c Nude Mice

This study was conducted in an identical manner to the study described in Example 1 with the following differences:

### 2. Experimental Design

| | | | | Dosing | | |
|---|---|---|---|---|---|---|
| | | | Dose | Volume | Dosing | |
| Group | Treatment | n | (mg/kg) | (ul/g) | Route | Schedule |
| 1 | Vehicle | 5 | — | 10 | iv | qw |
| 2 | BCY8242 | 3 | 3 mg/kg | 10 | iv | qw |
| 3 | BCY8242 | 3 | 3 mg/kg | 10 | iv | biw |
| 4 | BCY8242 | 3 | 5 mg/kg | 10 | iv | qw |

### 3.2 Test and Positive Control Articles

Product identification: BCY8242
Manufacturer: Bicycle Therapeutics
Lot number: 1
Physical description: Lyophilised powder
Molecular weight: 6346.46
Purity: 97.40%
Package and storage condition: stored at −80° C.

### 4.2 Tumor Inoculation

41 animals were randomized when the average tumor volume reached 162 mm³.

### 4.3 Testing Article Formulation Preparation

| Test | Con. | |
|---|---|---|
| article | (mg/ml) | Formulation |
| Vehicle | — | 25 mM Histidine pH 7 10% sucrose |
| BCY8242 | 20 | Dissolve 10.47 mg BCY8242 in 510 μl DMSO |
| BCY8242 | 0.5 | Dilute 510 μl 20 mg/ml BCY8242 stock with |
| | | 19.886 ml Histidine buffer |
| BCY8242 | 0.3 | Dilute 480 μl 0.5 mg/ml BCY8242 stock with |
| | | 320 μl Histidine buffer |

### 5. Results

### 5.1 Tumor Growth Curve

Tumor growth curves are shown in FIG. 8.

### 5.2 Tumor Volume Trace

Mean tumor volume over time in female Balb/c nude mice bearing NCI-H292 xenograft is shown in Table 3.

TABLE 3
Tumor volume trace over time

| | | Days after the start of treatment | | | | | | |
|---|---|---|---|---|---|---|---|---|
| Gr. | Treatment | 0 | 2 | 4 | 7 | 9 | 11 | 14 |
| 1 | Vehicle, qw | 161 ± 2 | 270 ± 14 | 357 ± 14 | 448 ± 17 | 570 ± 16 | 720 ± 36 | 948 ± 61 |
| 2 | BCY8242, | 160 ± 1 | 214 ± 13 | 197 ± 15 | 159 ± 2 | 175 ± 7 | 119 ± 8 | 92 ± 10 |
| | 3 mpk, qw | | | | | | | |
| 3 | BCY8242, | 162 ± 12 | 221 ± 9 | 176 ± 17 | 146 ± 36 | 131 ± 49 | 79 ± 25 | 73 ± 28 |
| | 3 mpk, biw | | | | | | | |
| 4 | BCY8242, | 163 ± 8 | 185 ± 18 | 133 ± 4 | 145 ± 18 | 131 ± 12 | 91 ± 5 | 81 ± 5 |
| | 5 mpk, qw | | | | | | | |

### 5.3 Tumor Growth Inhibition Analysis

Tumor growth inhibition rate for test articles in the NCI-H292 xenograft model was calculated based on tumor volume measurements at day 14 after the start of treatment.

TABLE 4
Tumor growth inhibition analysis

| | | Tumor | | | P value |
|---|---|---|---|---|---|
| | | Volume | T/C^{b} | TGI | compared |
| Gr | Treatment | (mm³)ª | (%) | (%) | with vehicle |
| 1 | Vehicle, qw | 948 ± 61 | — | — | — |
| 2 | BCY8242, | 92 ± 10 | 9.7 | 108.6 | p < 0.001 |
| | 3 mpk, qw | | | | |
| 3 | BCY8242, | 73 ± 28 | 7.7 | 111.4 | p < 0.001 |
| | 3 mpk, biw | | | | |
| 4 | BCY8242, | 81 ± 5 | 8.6 | 110.4 | p < 0.001 |
| | 5 mpk, qw | | | | |

^{a}Mean ± SEM.
^{b}Tumor Growth Inhibition is calculated by dividing the group average tumor volume for the treated group by the group average tumor volume for the control group (T/C).

### 6. Results Summary and Discussion

In this study, the therapeutic efficacy of BCY8242 in the NCI-H292 xenograft model was evaluated. The measured tumor volumes of all treatment groups at various time points are shown in the FIG. 3 and Tables 3 and 4.

The mean tumor size of vehicle treated mice reached 948 mm³ on day 14. BCY8242 at 3 mg/kg, qw (TV=92 mm³, TGI=108.6%, p<0.001), 3 mg/kg, biw (TV=73 mm³, TGI=111.4%, p<0.001) and 5 mg/kg, qw (TV=81 mm³, TGI=110.4%, p<0.001) produced significant antitumor activity.

1. A drug conjugate comprising:
(i) a peptide ligand comprising at least three reactive groups, separated by at least two loop sequences, and a non-aromatic molecular scaffold which forms covalent bonds with the reactive groups of the peptide ligand such that at least two polypeptide loops are formed on the molecular scaffold; and
(ii) two cytotoxic agents conjugated to said peptide ligand.

2. The drug conjugate as defined in claim 1, wherein said reactive groups comprise cysteine and/or (S)-2-amino-3-(methylamino)propanoic acid (Dap(Me)).

3. The drug conjugate as defined in claim 1 or claim 2, wherein said peptide ligand is specific for Nectin-4.

4. The drug conjugate as defined in claim 3, wherein said loop sequences comprise 3 or 9 amino acid acids.

5. The drug conjugate as defined in claim 3 or claim 4, wherein said peptide ligand has
(SEQ ID NO: 1)
CP(1Nal)(dD)CM(HArg)DWSTP(HyP)WC
wherein 1Nal represents 1-naphthylalanine, HArg represents homoarginine and HyP represents hydroxyproline.

6. The drug conjugate as defined in claim 3 or claim 4, wherein said peptide ligand has a core sequence of:
(SEQ ID NO: 2)
CP(1Nal)(dD)CMKDWSTP(HyP)WC,
wherein 1Nal represents 1-naphthylalanine and HyP represents hydroxyproline.

7. The drug conjugate as defined in any one of claims 1 to 6, wherein the molecular scaffold is 1,1′,1″-(1,3,5-triazinane-1,3,5-triyl)triprop-2-en-1-one (TATA).

8. The drug conjugate as defined in any one of claims 1 to 7, wherein the cytotoxic agent is (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide) (monomethyl auristatin E; MMAE) which has the following structure:

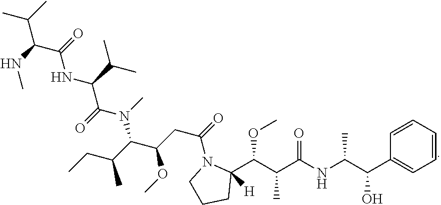 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

9. The drug conjugate as defined in any one of claims 1 to 8, which additionally comprises a linker between said peptide ligand and each of said cytotoxic agents.

10. The drug conjugate as defined in claim 9, wherein said linker is selected from one or more of: Val-Cit, β-Ala, p-aminobenzylcarbamate (PABC), glutaryl, N-(bis aminopropyl) glycine (BAPG) and one or more (e.g. 10) sarcosine (Sar) residues, such as: (i) a -Sar₁₀-βAla-BAPG-(glutaryl-Val-Cit-PABC)₂ linker, (ii) a -BAPG-(Sar₁₀-βAla-glutaryl-Val-Cit-PABC)₂ linker or a (iii) (Sar₁₀-βAla-glutaryl-Val-Cit-PABC)₂ linker.

11. The drug conjugate as defined in claim 1, which is selected from BCY8391, BCY8242 and BCY8243.

12. A pharmaceutical composition which comprises the drug conjugate of any one of claims 1 to 11, in combination with one or more pharmaceutically acceptable excipients.

13. The drug conjugate as defined in any one of claims 1 to 11, for use in preventing, suppressing or treating diseases.

14. The drug conjugate for use as defined in claim 13, wherein said disease is one which may be alleviated by cell death

15. The drug conjugate for use as defined in claim 14, wherein said disease is selected from diseases characterised by defective cell types, proliferative disorders such as cancer and autoimmune disorders such as rheumatoid arthritis.
